(19) United States
(12) Patent Application Publication
(10) Pub. No.: US 2018/0235177 A1
(43) Pub. Date: Aug. 23, 2018

(54) Post-surgery Protective Garment for Preventing an Animal from Biting or Licking Incision Areas

(71) Applicant: Botanical Designs, LLC, Northbrook, IL (US)

(72) Inventor: Julie Gorden, Riverwoods, IL (US)

(21) Appl. No.: 15/900,781

(22) Filed: Feb. 20, 2018

Related U.S. Application Data
(60) Provisional application No. 62/460,591, filed on Feb. 17, 2017.

(51) Int. Cl. A01K 13/00; A61F 15/00

(52) U.S. Cl. CPC A01K 13/007; A61F 15/008

(57) ABSTRACT

A post-surgery protective garment is worn by an animal to prevent the animal from biting or licking incision areas, until those incision areas are completely healed on the animal. The post-surgery protective garment includes a torso-covering sheet, a left placket, a right placket, and a plurality of placket fasteners. The torso-covering sheet encloses the torso of a four-legged animal and covers incision wounds made on the torso. The left placket and the right placket secures the torso-covering sheet onto the animal. The plurality of placket fasteners attaches the left placket to the right placket. More specifically, the plurality of placket fasteners positions the left placket and the right placket to overlap each other. By adjusting the overlap between the left placket and the right placket, girth of the torso-covering sheet can be widened to fit larger animals.

The current application claims a priority to the U.S. Provisional Patent application Ser. No. 62/460,591 filed on Feb. 17, 2017. The current application is filed on the next business day, which is Feb. 20, 2018, while Feb. 17, 2018 was on a weekend and Feb. 19, 2018 was on a national holiday (President's Day).

### FIELD OF THE INVENTION

The present invention generally relates to a post-surgery protective garment for preventing an animal from biting or licking incision areas made during a surgery. More specifically, the present invention utilizes a torso-covering sheet including a front pair of leg holes and a rear pair of leg holes to cover the whole body of the animal and to allow the animal to move about.

### BACKGROUND OF THE INVENTION

Animals may have to undergo surgery at some points in their lifespan. After surgery, the animal may require a cone to be placed around the head of the animal preventing the animal from biting/picking at the surgical points. The cone can be very cumbersome to the animal. Animals are typically uncomfortable with cone in addition to the discomfort from the surgical incisions. The cone makes it difficult for animals to perform daily actions such as moving around, sleeping, and eating.

It is therefore an objective of the present invention to provide a protective garment for animals, which helps prevent them from irritating post-surgical incisions or areas of the skin due to itching from allergies or hot spots. The present invention serves as a comfortable alternative to a cone. The present invention includes openings for the body and paws of the animal. The present invention provides a secure fit with a plurality of fasteners. The present invention includes a flap that covers the lower body and rear of the animal. The flap can be opened and closed with a plurality of flap fasteners. The animal can urinate and/or defecate when the flap is open. The plurality of fasteners is positioned strategically to prevent the animals from disengaging the fasteners and ultimately removing the present invention. The animal will be more comfortable using the present invention rather than a cone and still be prevented from irritating post-surgical incisions or areas of the skin due to itching from allergies or hot spots.

### BRIEF DESCRIPTION OF THE DRAWINGS

FIG. 1 is a bottom view of the present invention illustrating the belly flap.

FIG. 2 is a top view of the present invention illustrating the left placket separated from the right placket while the present invention is worn by a dog.

FIG. 3 is a bottom view illustrating the belly flap in the closed position.

FIG. 4 is a bottom view illustrating the belly flap in the open position.

FIG. 5 is a top view illustrating the left placket engaged to the right placket.

FIG. 6 is a side view illustrating the front pair of leg sleeves and the rear pair of leg sleeves.

FIG. 7 is a front view of the hood.

FIG. 8 is a top view of the front pair of paw covers.

FIG. 9 is a bottom view of the rear pair of paw covers.

### DETAILED DESCRIPTION OF THE INVENTION

All illustrations of the drawings are for the purpose of describing selected versions of the present invention and are not intended to limit the scope of the present invention.

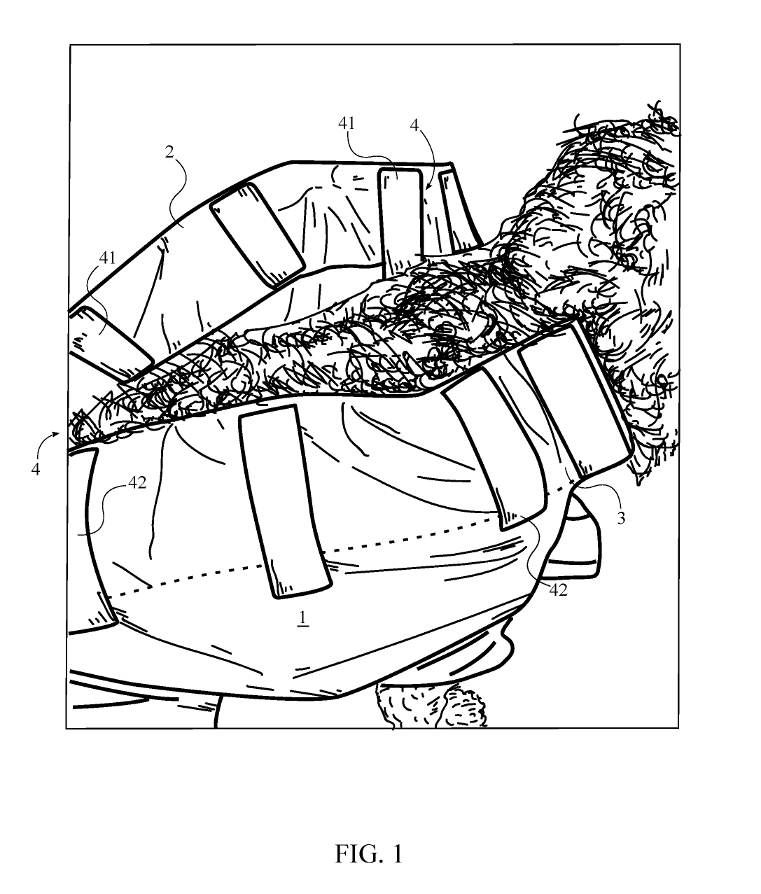

The present invention is a one-piece protective garment used for post-surgery recovery process that deters animals from biting or licking incisions made during surgery. Referring to FIG. 1, the preferred embodiment of the present invention comprises a torso-covering sheet 1, a left placket 2, a right placket 3, and a plurality of placket fasteners 4. The torso-covering sheet 1 is made of a stretchable fabric specially designed so that the torso-covering sheet 1 does not unravel when cut. In the preferred embodiment, the present invention is designed to be worn by a four-legged animal such as a dog, a cat, or similar animals that are usually kept as pets. As such, the torso-covering sheet 1 encloses the torso of the animal. The left placket 2 and the right placket 3 secure the torso-covering sheet 1 to the animal's body. More specifically, the left placket 2 connects to the right placket 3 over the animal's back, thus securely enclosing the torso of the animal within the torso-covering sheet 1. The plurality of placket fasteners 4 releasably fastens the left placket 2 to the right placket 3. The left placket 2 can thus be detached from the right placket 3 to create an opening whereby the animal can slip into and out of the torso-covering sheet 1.

In the preferred embodiment of the present invention, the torso-covering sheet 1 is made of a waterproof fabric. Further, the fabric is also lightweight, comfortable, breathable, moisture-wicking, and dries quickly.

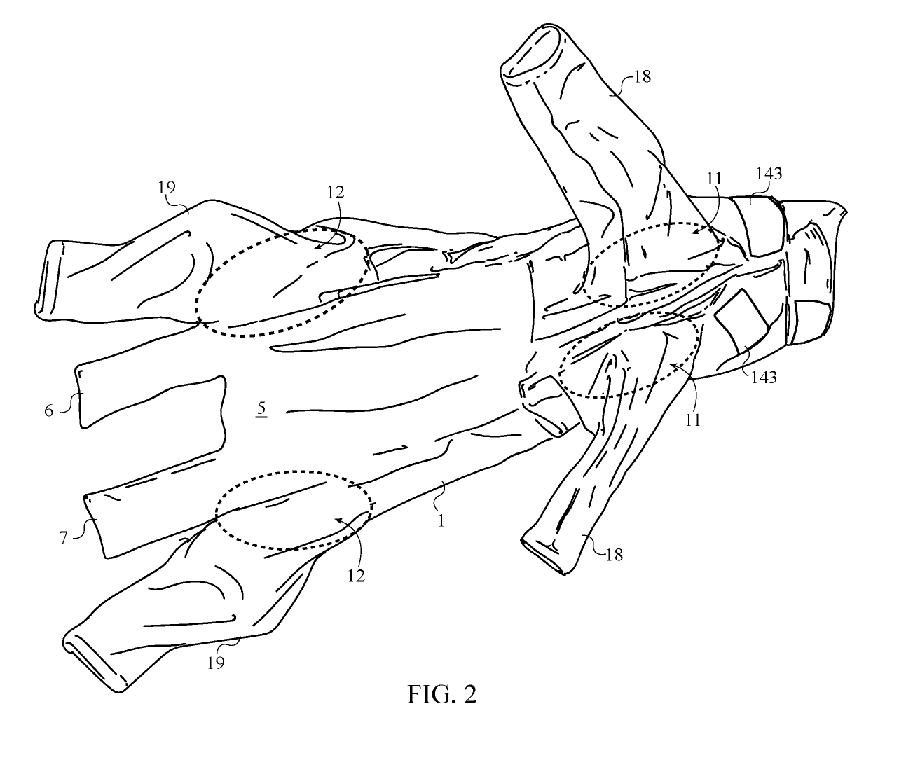

Referring to FIG. 2, generally, the animal must wear the torso-covering sheet 1 until the incision wounds have completely healed, and the present invention is thus designed accommodate the animal's daily activities. Accordingly, the torso-covering sheet 1 comprises a front pair of leg holes 11 and a rear pair of leg holes 12 that allows the animal to remain mobile while wearing the present invention. Each of the front pair of leg holes 11 braces the upper arm of the animal, whereas each of the rear pair of leg holes 12 braces the upper thigh of the animal. Once the legs are inserted into the front pair of leg holes 11 and the rear pair of leg holes 12, the plurality of placket fasteners 4 secures the torso-covering sheet 1 around the animal. As such, each of the plurality of placket fasteners 4 comprises a first placket-interlocking portion 41 and a second placket-interlocking portion 42. Preferably, the plurality of placket fasteners 4 is a hook-and-loop fastener. Alternately, the plurality of placket fasteners 4 may utilize zippers, snap fasteners, buttons, and the like.

The left placket 2 is connected adjacent to the torso-covering sheet 1. Similarly, the right placket 3 is also connected adjacent to the torso-covering sheet 1, opposite to the left placket 2. Preferably, both the left placket 2 and the right placket 3 are formed by folding an edge of the torso-covering sheet 1 and stitching two layers together. This increases the rigidity of the left placket 2 and the right placket 3 and allowing the mounting of the plurality of placket fasteners 4. More specifically, the first placket-interlocking portion 41 for each of the plurality of placket fasteners 4 is connected along the left placket 2. Moreover, the second placket-interlocking portion 42 for each of the plurality of placket fasteners 4 is connected along the right placket 3. Preferably, the first placket-interlocking portion 41 is the hook portion of a hook-and-loop fastener, whereas the second placket-interlocking portion 42 is the loop portion of the hook-and-loop fastener. As such, the left placket 2 must overlap the right placket 3 to successfully create an interlocking connection. Alternately, the first placket-interlocking portion 41 may be the loop portion of a hook-and-loop fastener, whereas the second placket-interlocking portion 42 may be the hook portion of the hook-and-loop fastener. In this case, the right placket 3 must overlap the left placket 2 to create an interlocking connection.

Further, the plurality of placket fasteners 4 allows the girth of the torso-covering sheet 1 to be adjusted. As such, the present invention can be adjusted for animals with different sized necks. More specifically, the overlap distance of the left placket 2 over the right placket 3 or the right placket 3 over the left placket 2 can be adjusted. For example, increasing the overlap between the left placket 2 and the right placket 3 would narrow the girth of the torso-covering sheet 1. Alternately, reducing the overlap between the left placket 2 and the right placket 3 would widen the girth of the torso-covering sheet 1.

In the preferred implementation, the front legs and the rear legs are afforded their full range of motion. Once the legs insert into the front pair of leg holes 11 and the rear pair of leg holes 12, the left placket 2 and the right placket 3 are engaged together by the plurality of placket fasteners 4. As such, the front pair of leg holes 11 and the rear pair of leg holes 12 are positioned in between the left placket 2 and the right placket 3. Further, the front pair of leg holes 11 and the rear pair of leg holes 12 are positioned opposite to each other across the torso-covering sheet 1. Preferably, the front legs and the rear legs of animal are vertically inserted into the front pair of leg holes 11 and the rear pair of leg holes 12, respectively.

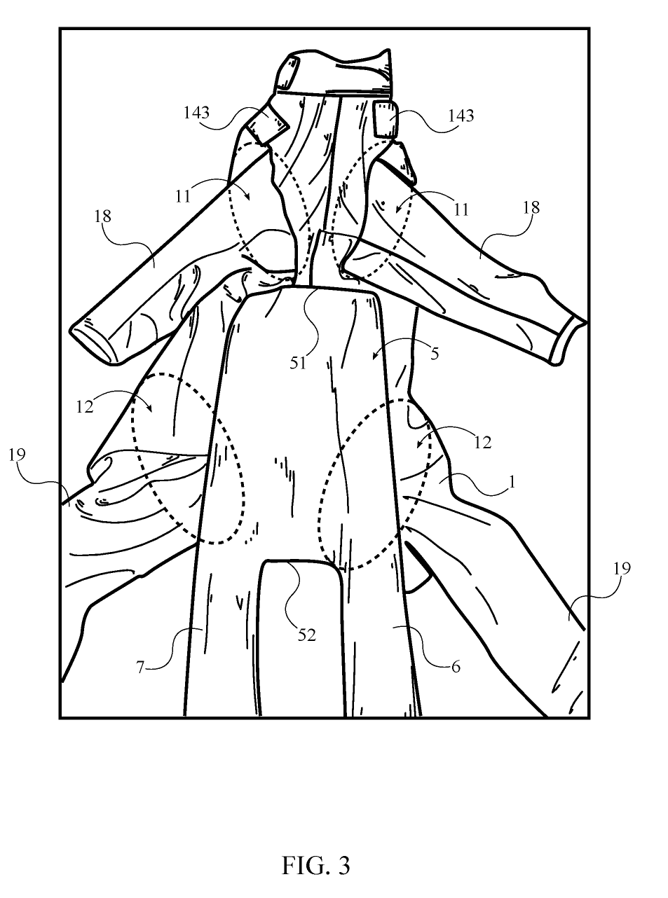

Referring to FIG. 3, the underbody of the animal is covered by a belly flap 5 that opens to allow the animal to defecate and urinate while wearing the present invention. A left bracing strap 6 and a right bracing strap 7 secures the belly flap 5 in either the opened position or closed position. The torso-covering sheet 1 further comprises a rear access gap 13 that exposes the underbody of the animal, while the belly flap 5 is secured in the opened position. This allows the animal to defecate and urinate without taking off the torso-covering sheet 1. Preferably, the belly flap 5 comprises a proximal edge 51 and a distal edge 52. Further, the rear access gap 13 being positioned in between the rear pair of leg holes 12. The length of the belly flap is determined by the distance between the proximal edge 51 and the distal edge 52. Generally, the belly flap 5 is sized to completely cover the rear access gap 13. The proximal edge 51 is hingedly connected to the torso-covering sheet 1, adjacent to the rear access gap 13. More specifically, the proximal edge 51 is sewn onto the rear access gap 13 in a manner which allows the belly flap 5 to completely cover the rear access gap 13. The left bracing strap 6 and the right bracing strap 7 is connected adjacent to the distal edge 52. Further, the left bracing strap 6 and the right bracing strap 7 is positioned opposite to each other along the distal edge 52. To secure the belly flap 5 in the open position, the left bracing strap 6 and the right bracing strap 7 are folded over and positioned proximal to the animal's neck. In the closed position, the left bracing strap 6 and the right bracing strap 7 are positioned near the haunch of the animal.

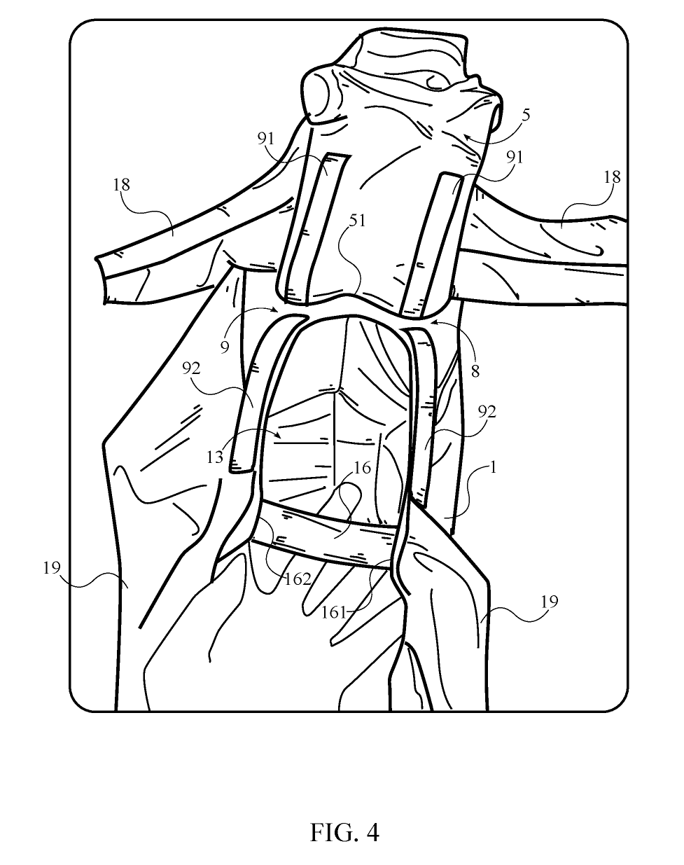

Referring to FIG. 4, a left flap fastener 8 and a right flap fastener 9 attaches the belly flap 5 to the torso-covering sheet 1. The left flap fastener 8 and the right flap fastener 9 each comprise a first flap-interlocking portion 91 and a second flap-interlocking portion 92. Preferably, the first flap-interlocking portion 91 and the second flap-interlocking portion 92 are either the hook portion or the loop portion of a hook-and-loop fastener, respectively. Alternately, the first flap-interlocking portion 91 and the second flap-interlocking portion 92 may be any releasable fasteners. The first flap-interlocking portion 91 of the left flap fastener 8 and the first flap-interlocking portion 91 of the right flap fastener 9 are both mounted onto the belly flap 5. Preferably, the first flap-interlocking portion 91 of the left flap fastener 8 and the first flap-interlocking portion 91 of the right flap fastener 9 are positioned opposite to each other across the belly flap 5. More specifically, the left flap fastener 8 is mounted adjacent to a left edge of the belly flap 5, whereas the right flap fastener 9 is mounted adjacent to right edge of the belly flap 5. In contrast, the second flap-interlocking portion 92 of the left flap fastener 8 and the second flap-interlocking portion 92 of the right flap fastener 9 are mounted onto the torso-covering sheet 1. Further, the second flap-interlocking portion 92 of the left flap fastener 8 and the second flap-interlocking portion 92 of the right flap fastener 9 are positioned opposite to each other across the rear access gap 13. To close the belly flap 5, the first flap-interlocking portion 91 of the left flap fastener 8 and the second flap-interlocking portion 92 of the left flap fastener 8 are positioned coincident to each other. Similarly, the first flap-interlocking portion 91 of the right flap fastener 9 and the second flap-interlocking portion 92 of the right flap fastener 9 are positioned coincident to each other.

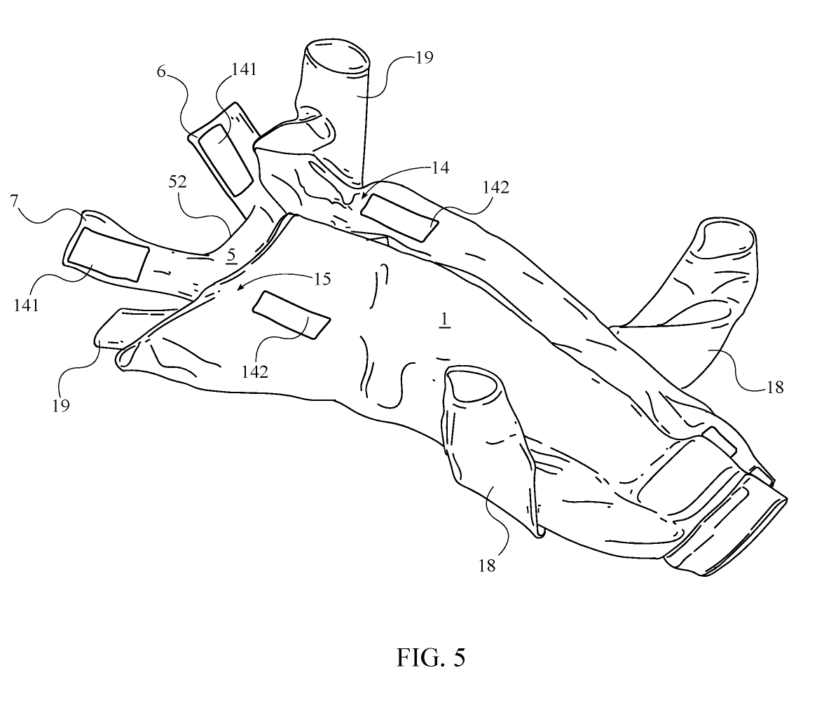

Referring to FIG. 3, FIG. 4, and FIG. 5, a left strap fastener 14 attaches the left bracing strap 6 to the torso-covering sheet 1. As such, the left strap fastener 14 comprises a first strap-interlocking portion 141, a second strap-interlocking portion 142, and a third strap-interlocking portion 143. The first strap-interlocking portion 141 and the second strap-interlocking portion 142 are preferably the hook portion or the loop portion of a hook-and-loop fastener. The first strap-interlocking portion 141 is mounted onto the left bracing strap 6, opposite the distal edge 52. In contrast, the second strap-interlocking portion 142 and the third strap-interlocking portion 143 are mounted onto the torso-covering sheet 1. In the closed position, the belly flap 5 is positioned so that the first strap-interlocking portion 141 is positioned coincident to the second strap-interlocking portion 142. As such, the second strap-interlocking portion 142 is positioned in between the left placket 2 and the rear pair of leg holes 12. In the opened position, the belly flap 5 folds in front of the rear access gap 13, thereby positioning the first strap-interlocking portion 141 coincident to the third strap-interlocking portion 143. As such, the third strap-interlocking portion 143 is positioned in between the left placket 2 and the front pair of leg holes 11.

Likewise, a right strap fastener 15 attaches the right bracing strap 7 to the torso-covering sheet 1. As such, the right strap fastener 15 comprises a first strap-interlocking portion 141, a second strap-interlocking portion 142, and a third strap-interlocking portion 143. The first strap-interlocking portion 141 and the second strap-interlocking portion 142 are preferably either the hook portion or the loop portion of a hook-and-loop fastener. The first strap-interlocking portion 141 is mounted onto the right bracing strap 7, opposite the distal edge 52. In contrast, the second strap-interlocking portion 142 and the third strap-interlocking portion 143 are mounted onto the torso-covering sheet 1. In the closed position, the belly flap 5 is positioned so that the first strap-interlocking portion 141 is positioned coincident to the second strap-interlocking portion 142. As such, the second strap-interlocking portion 142 is positioned in between the right placket 3 and the rear pair of leg holes 12. In the opened position, the belly flap 5 folds in front of the rear access gap 13, thereby positioning the first strap-interlocking portion 141 coincident to the third strap-interlocking portion 143. As such, the third strap-interlocking portion 143 is positioned in between the right placket 3 and the front pair of leg holes 11.

In an alternate embodiment of the present invention, the left strap fastener 14 and the right strap fastener 15 may each comprise a series of strap-interlocking portion 142. The series of strap-interlocking portion 142 of the left strap fastener 14 may be distributed along the left placket 2. Similarly, the series of strap-interlocking portion 142 of the right strap fastener 15 may be distributed along the right placket 3. As such, the first strap-interlocking portion 141 of the left strap fastener 14 and the right strap fastener 15 can attach to different positions on the torso-covering sheet 1. For example, the length of the torso-covering sheet 1 is shortened by attaching the first strap-interlocking portion 141 of the left strap fastener 14 and the right strap fastener 15 to one of the series of strap-interlocking portion 142 positioned closer to the front pair of leg holes 11. Similarly, the torso-covering sheet 1 can be lengthened by attaching the first strap-interlocking portion 141 of the left strap fastener 14 and the right strap fastener 15 to one of the series of strap-interlocking portion 142 positioned closer to the rear pair of leg holes 12.

Referring back to FIG. 4, a tensioning band 16 helps maintain the rear pair of leg holes 12 in close proximity. As such, the tensioning band 16 comprises a first band end 161 and a second band end 162. The first band end 161 and the second band end 162 are mounted onto the torso-covering sheet 1. Further, the tensioning band 16 is positioned across the rear access gap 13. The tensioning band 16 keeps the rear pair of leg holes 12 close together. In the preferred implementation, the first band end 161 and the second band end 162 are stitched onto the portions of the torso-covering sheet 1 surrounding the rear access gap 13. As such, the tensioning band 16 ensures the rear pair of leg holes 12 remain close to each other.

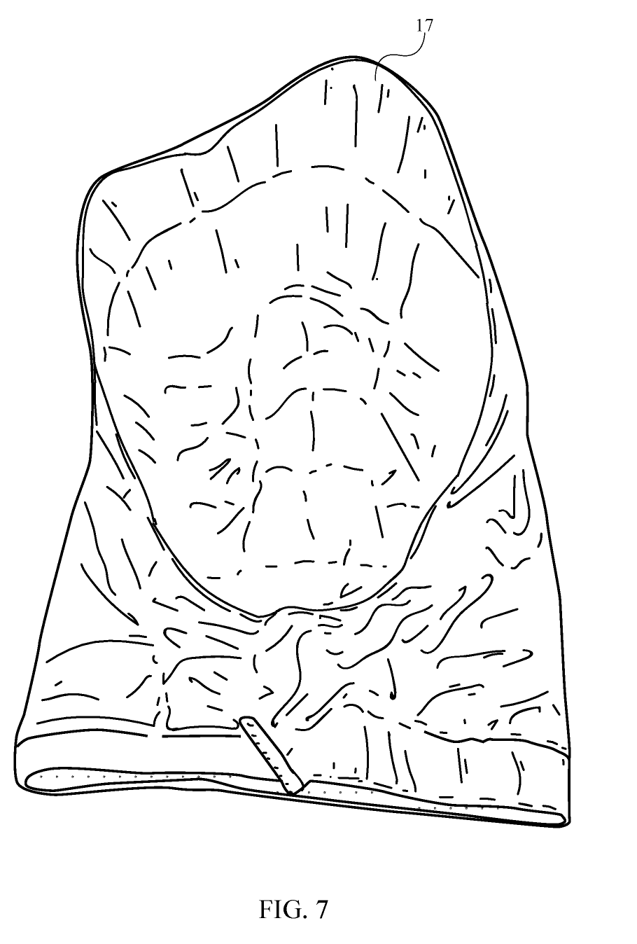

Referring to FIG. 7, a detachable hood 17 can be mounted onto the torso-covering sheet 1 to cover the animal's head. In particular, the hood 17 can be used to cover incision wounds on the head or neck of the animal. As such, the hood 17 is attached adjacent to the torso-covering sheet 1. Further, the hood 17 is positioned adjacent to the front pair of leg holes 11. In the preferred implementation, the collar of the torso-covering sheet 1 is fitted with either the hook portion or the loop portion of a hook-and-loop fastener. Accordingly, the hood is also fitted with the hook portion or the loop portion of a hook-and-loop fastener, whereby the hood is attached to the collar of the torso-covering sheet 1. This positions the hood 17 in front of the torso-covering sheet 1 and over the head of the animal.

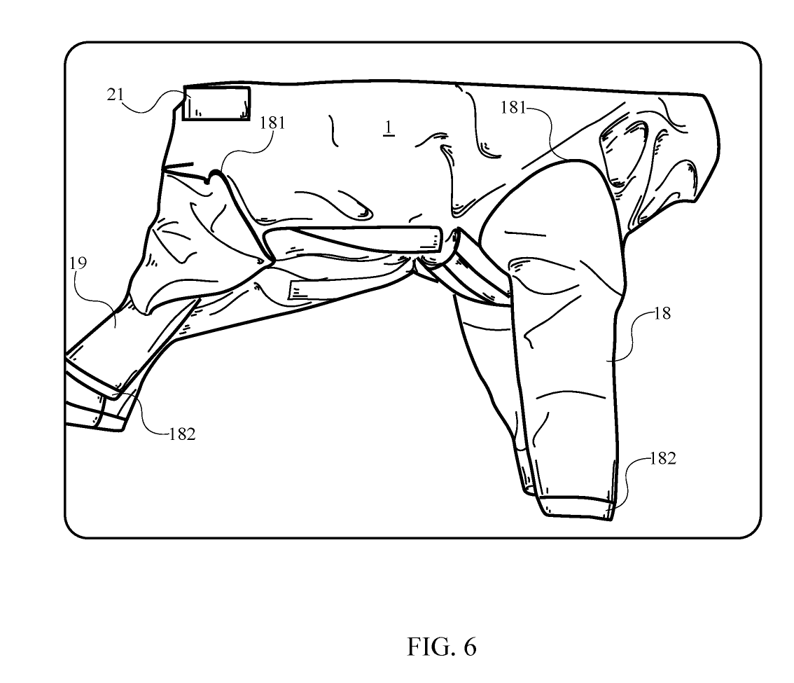

Referring to FIG. 6, a front pair of leg sleeves 18 covers the front legs and prevents the animal from agitating incision wounds made on the legs. Each of the front pair of leg sleeves 18 comprises a proximal rim 181 and a distal rim 182. The proximal rim 181 for each of the front pair of leg sleeves 18 is mounted onto the torso-covering sheet 1. Further, the proximal rim 181 for each of the front pair of leg sleeves 18 is positioned around a corresponding hole from the front pair of leg holes 11. More specifically, the proximal rim 181 is positioned around the upper arm of the animal. This affords the full range of motion to the animal's front legs.

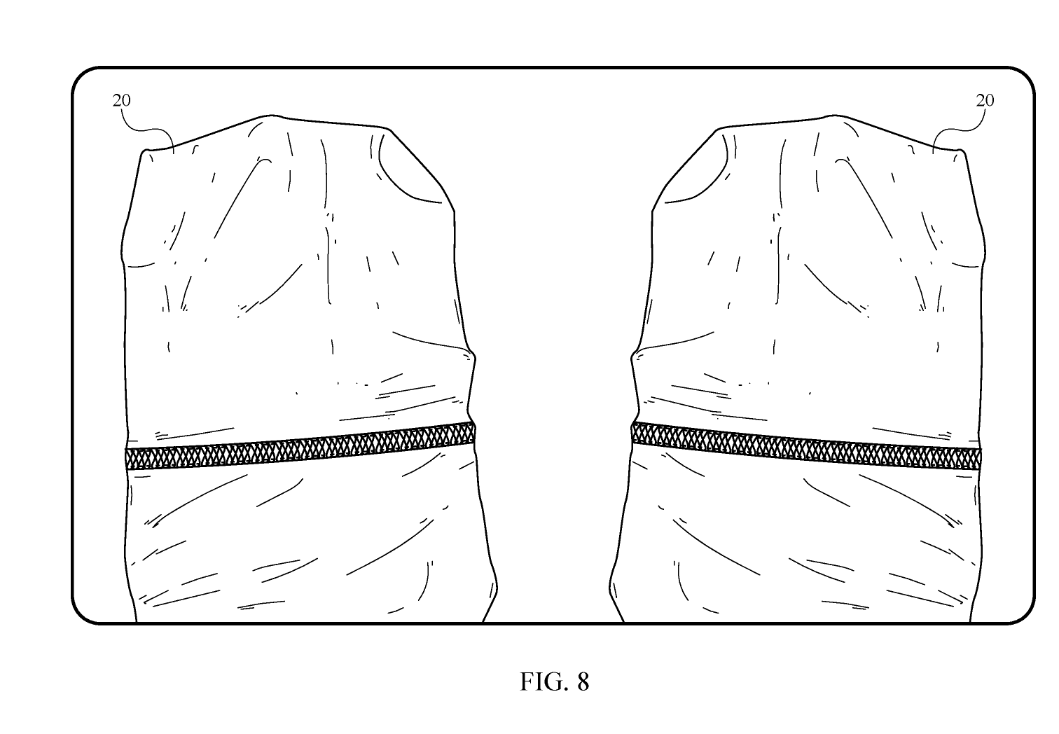

Referring to FIG. 8, a front pair of paw covers 20 is provided to protect the paws of the animal. The distal rim 182 for each of the front pair of leg sleeves 18 is perimetrically attached to a corresponding cover from the front pair of paw covers 20. Each of the front pair of paw covers 20 is releasably attached to the distal rim 182 via zippers, hook-and-loop fasteners, snaps, or similar mechanisms.

Referring back to FIG. 6, a rear pair of leg sleeves 19 covers the rear legs and prevents the animal from agitating incision wounds made on the legs. Each of the rear pair of leg sleeves 19 comprises a proximal rim 181 and a distal rim 182. The proximal rim 181 for each of the rear pair of leg sleeves 19 is mounted onto the torso-covering sheet 1. Further, the proximal rim 181 for each of the rear pair of leg sleeves 19 is positioned around a corresponding hole from the rear pair of leg holes 12. More specifically, the proximal rim 181 is positioned around the upper thigh of the animal affording the full range of motion to the animal's rear legs.

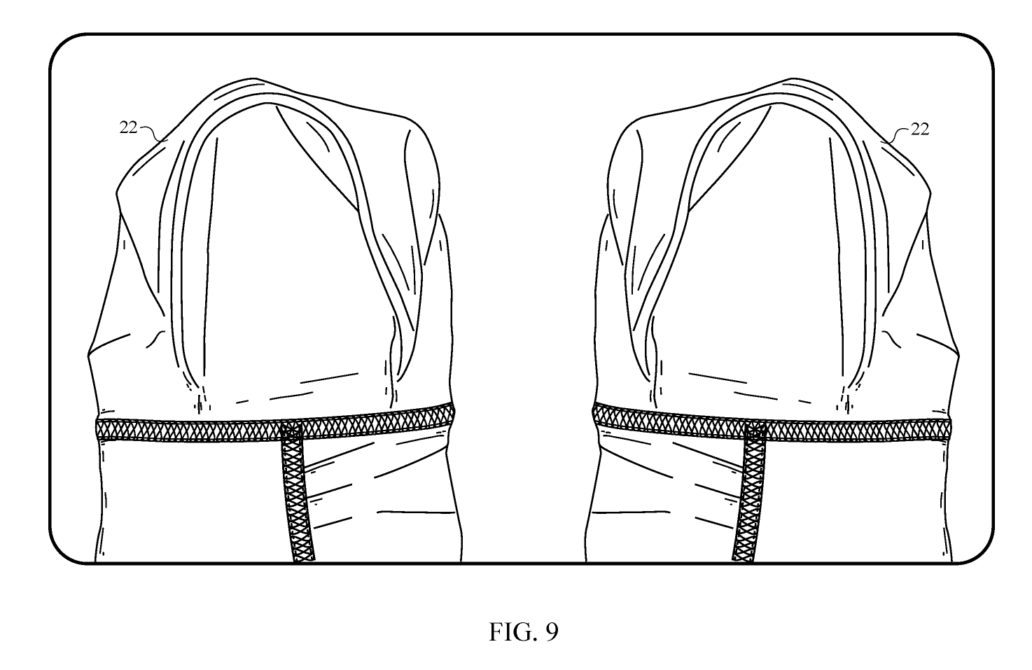

Referring to FIG. 9, a rear pair of paw covers 22 is provided to protect the paws of the animal. The distal rim 182 for each of the rear pair of leg sleeves 18 is perimetrically attached to a corresponding cover from the rear pair of paw covers 22. Each of the rear pair of paw covers 22 is releasably attached to the distal rim 182 via zippers, hook-and-loop fasteners, snaps, or similar mechanisms.

Referring back to FIG. 6, the lengths of both the front pair of leg sleeves 18 and rear pair of leg sleeves 19 can be adjusted to fit the height of the animal. Since the front pair of leg sleeves 18 and the rear pair of leg sleeves 19 are not closed or cuffed, the ends of the front pair of leg sleeves 18 and the rear pair of leg sleeves 19 can be cut or rolled for animals with shorter legs. Accordingly, the front pair of leg sleeves 18 and the rear pair of leg sleeves 19 are made of a fabric that does not unravel when cut.

Finally, at least one night reflector 21 to increase the visibility of the torso-covering sheet 1 while in low visibility areas or during the night. This allows the animal to take a walk at night while wearing the torso-covering sheet 1. The night reflector 21 is mounted onto the torso-covering sheet 1. Preferably, the night reflector 21 is a sticker made of glow-in-the-dark materials that may be attached to the torso-covering sheet 1. Alternately, the night reflector 21 may be made by painting sections of the torso-covering sheet 1 with high-visibility paints.

Although the invention has been explained in relation to its preferred embodiment, it is to be understood that many other possible modifications and variations can be made without departing from the spirit and scope of the invention as hereinafter claimed.

What is claimed is:

1. A post-surgery protective garment for preventing an animal from biting or licking incision areas comprises:
a torso-covering sheet;
a left placket;
a right placket;
a plurality of placket fasteners;
the torso-covering sheet comprises a front pair of leg holes and a rear pair of leg holes;
each of the plurality of placket fasteners comprises a first placket-interlocking portion and a second placket-interlocking portion;
the left placket being connected adjacent to the torso-covering sheet;
the right placket being connected adjacent to the torso-covering sheet, opposite to the left placket;
the first placket-interlocking portion for each of the plurality of placket fasteners being connected along the left placket;
the second placket-interlocking portion for each of the plurality of placket fasteners being connected along the right placket;
the front pair of leg holes and the rear pair of leg holes being positioned in between the left placket and the right placket; and
the front pair of leg holes and the rear pair of leg holes being positioned opposite to each other across the torso-covering sheet.

2. The post-surgery protective garment for preventing an animal from biting or licking incision areas as claimed in claim 1 comprises:
a belly flap;
a left bracing strap;
a right bracing strap;
the torso-covering sheet further comprises a rear access gap;
the belly flap comprises a proximal edge and a distal edge;
the rear access gap being positioned in between the rear pair of leg holes;
the proximal edge being hingedly connected to the torso-covering sheet, adjacent to the rear access gap;
the left bracing strap and the right bracing strap being connected adjacent to the distal edge; and
the left bracing strap and the right bracing strap being positioned opposite to each other along the distal edge.

3. The post-surgery protective garment for preventing an animal from biting or licking incision areas as claimed in claim 2 comprises:
a left flap fastener;
a right flap fastener;
the left flap fastener and the right flap fastener each comprise a first flap-interlocking portion and a second flap-interlocking portion;
the first flap-interlocking portion of the left flap fastener and the first flap-interlocking portion of the right flap fastener being mounted onto the belly flap;
the first flap-interlocking portion of the left flap fastener and the first flap-interlocking portion of the right flap fastener being positioned opposite to each other across the belly flap;
the second flap-interlocking portion of the left flap fastener and the second flap-interlocking portion of the right flap fastener being mounted onto the torso-covering sheet; and
the second flap-interlocking portion of the left flap fastener and the second flap-interlocking portion of the right flap fastener being positioned opposite to each other across the rear access gap.

4. The post-surgery protective garment for preventing an animal from biting or licking incision areas as claimed in claim 2 comprises:
a left strap fastener;
the left strap fastener comprises a first strap-interlocking portion, a second strap-interlocking portion, and a third strap-interlocking portion;
the first strap-interlocking portion being mounted onto the left bracing strap, opposite the distal edge;
the second strap-interlocking portion and the third strap-interlocking portion being mounted onto the torso-covering portion;
the second strap-interlocking portion being positioned in between the left placket and the rear pair of leg holes; and
the third strap-interlocking portion being positioned in between the left placket and the front pair of leg holes.

5. The post-surgery protective garment for preventing an animal from biting or licking incision areas as claimed in claim 2 comprises:
a right strap fastener;
the right fastener comprises a first strap-interlocking portion, a second strap-interlocking portion, and a third strap-interlocking portion;
the first strap-interlocking portion being mounted onto the right bracing strap, opposite the distal edge;
the second strap-interlocking portion and the third strap-interlocking portion being mounted onto the torso-covering portion;
the second strap-interlocking portion being positioned in between the right placket and the rear pair of leg holes; and
the third strap-interlocking portion being positioned in between the right placket and the front pair of leg holes.

6. The post-surgery protective garment for preventing an animal from biting or licking incision areas as claimed in claim 2 comprises:
a tensioning band;
the tensioning band comprises a first band end and a second band end;
the first band end and the second band end being mounted onto the torso-covering portion; and
the tensioning band being positioned across the rear access gap.

7. The post-surgery protective garment for preventing an animal from biting or licking incision areas as claimed in claim 1 comprises:
a hood;
the hood being attached adjacent to the torso-covering sheet; and
the hood being positioned adjacent to the front pair of leg holes.

8. The post-surgery protective garment for preventing an animal from biting or licking incision areas as claimed in claim 1 comprises:
a front pair of leg sleeves;
each of the front pair of leg sleeves comprises a proximal rim and a distal rim;
the proximal rim for each of the front pair of leg sleeves being mounted onto the torso-covering portion; and
the proximal rim for each of the front pair of leg sleeves being positioned around a corresponding hole from the front pair of leg holes.

9. The post-surgery protective garment for preventing an animal from biting or licking incision areas as claimed in claim 8 comprises:
a front pair of paw covers; and
the distal rim for each of the front pair of leg sleeves being perimetrically attached to a corresponding cover from the front pair of paw covers.

10. The post-surgery protective garment for preventing an animal from biting or licking incision areas as claimed in claim 1 comprises:
a rear pair of leg sleeves;
each of the rear pair of leg sleeves comprises a proximal rim and a distal rim;
the proximal rim for each of the rear pair of leg sleeves being mounted onto the torso-covering portion; and
the proximal rim for each of the rear pair of leg sleeves being positioned around a corresponding hole from the rear pair of leg holes.

11. The post-surgery protective garment for preventing an animal from biting or licking incision areas as claimed in claim 10 comprises:
a rear pair of paw covers; and
the distal rim for each of the rear pair of leg sleeves being perimetrically attached to a corresponding cover from the rear pair of paw covers.

12. The post-surgery protective garment for preventing an animal from biting or licking incision areas as claimed in claim 1 comprises:
at least one night reflector; and
the night reflector being mounted onto the torso-covering sheet.

13. The post-surgery protective garment for preventing an animal from biting or licking incision areas as claimed in claim 1, wherein the torso-covering sheet is made of a waterproof fabric.

14. A post-surgery protective garment for preventing an animal from biting or licking incision areas comprises:
a torso-covering sheet;
a left placket;
a right placket;
a plurality of placket fasteners;
a belly flap;
a left bracing strap;
a right bracing strap;
the torso-covering sheet comprises a front pair of leg holes and a rear pair of leg holes;
each of the plurality of placket fasteners comprises a first placket-interlocking portion and a second placket-interlocking portion;
the torso-covering sheet further comprises a rear access gap;
the belly flap comprises a proximal edge and a distal edge;
the left placket being connected adjacent to the torso-covering sheet;
the right placket being connected adjacent to the torso-covering sheet, opposite to the left placket;
the first placket-interlocking portion for each of the plurality of placket fasteners being connected along the left placket;
the second placket-interlocking portion for each of the plurality of placket fasteners being connected along the right placket;
the front pair of leg holes and the rear pair of leg holes being positioned in between the left placket and the right placket;
the front pair of leg holes and the rear pair of leg holes being positioned opposite to each other across the torso-covering sheet;
the rear access gap being positioned in between the rear pair of leg holes;
the proximal edge being hingedly connected to the torso-covering sheet, adjacent to the rear access gap;
the left bracing strap and the right bracing strap being connected adjacent to the distal edge;
the left bracing strap and the right bracing strap being positioned opposite to each other along the distal edge; and
the torso-covering sheet being made of a waterproof fabric.

15. The post-surgery protective garment for preventing an animal from biting or licking incision areas as claimed in claim 14 comprises:
a left flap fastener;
a right flap fastener;
the left flap fastener and the right flap fastener each comprise a first flap-interlocking portion and a second flap-interlocking portion;
the first flap-interlocking portion of the left flap fastener and the first flap-interlocking portion of the right flap fastener being mounted onto the belly flap;
the first flap-interlocking portion of the left flap fastener and the first flap-interlocking portion of the right flap fastener being positioned opposite to each other across the belly flap;
the second flap-interlocking portion of the left flap fastener and the second flap-interlocking portion of the right flap fastener being mounted onto the torso-covering sheet; and
the second flap-interlocking portion of the left flap fastener and the second flap-interlocking portion of the right flap fastener being positioned opposite to each other across the rear access gap.

16. The post-surgery protective garment for preventing an animal from biting or licking incision areas as claimed in claim 14 comprises:
a left strap fastener;
a right strap fastener;
the left strap fastener comprises a first strap-interlocking portion, a second strap-interlocking portion, and a third strap-interlocking portion;
the right fastener comprises a first strap-interlocking portion, a second strap-interlocking portion, and a third strap-interlocking portion;
the first strap-interlocking portion being mounted onto the left bracing strap, opposite the distal edge;
the second strap-interlocking portion and the third strap-interlocking portion being mounted onto the torso-covering portion;
the second strap-interlocking portion being positioned in between the left placket and the rear pair of leg holes;
the third strap-interlocking portion being positioned in between the left placket and the front pair of leg holes;
the first strap-interlocking portion being mounted onto the right bracing strap, opposite the distal edge;
the second strap-interlocking portion and the third strap-interlocking portion being mounted onto the torso-covering portion;
the second strap-interlocking portion being positioned in between the right placket and the rear pair of leg holes; and
the third strap-interlocking portion being positioned in between the right placket and the front pair of leg holes.

17. The post-surgery protective garment for preventing an animal from biting or licking incision areas as claimed in claim 14 comprises:
a tensioning band;
the tensioning band comprises a first band end and a second band end;
the first band end and the second band end being mounted onto the torso-covering portion; and
the tensioning band being positioned across the rear access gap.

18. The post-surgery protective garment for preventing an animal from biting or licking incision areas as claimed in claim 14 comprises:
a hood;
the hood being attached adjacent to the torso-covering sheet; and
the hood being positioned adjacent to the front pair of leg holes.

19. The post-surgery protective garment for preventing an animal from biting or licking incision areas as claimed in claim 14 comprises:
a front pair of leg sleeves;
a front pair of paw covers;
each of the front pair of leg sleeves comprises a proximal rim and a distal rim;
the proximal rim for each of the front pair of leg sleeves being mounted onto the torso-covering portion;
the proximal rim for each of the front pair of leg sleeves being positioned around a corresponding hole from the front pair of leg holes; and
the distal rim for each of the front pair of leg sleeves being perimetrically attached to a corresponding cover from the front pair of paw covers.

20. The post-surgery protective garment for preventing an animal from biting or licking incision areas as claimed in claim 14 comprises:
a rear pair of leg sleeves;
a rear pair of paw covers;
at least one night reflector;
each of the rear pair of leg sleeves comprises a proximal rim and a distal rim;
the proximal rim for each of the rear pair of leg sleeves being mounted onto the torso-covering portion;
the proximal rim for each of the rear pair of leg sleeves being positioned around a corresponding hole from the rear pair of leg holes;
the distal rim for each of the rear pair of leg sleeves being perimetrically attached to a corresponding cover from the rear pair of paw covers; and
the night reflector being mounted onto the torso-covering sheet.
